(19) United States
(12) Patent Application Publication
(10) Pub. No.: US 2024/0415497 A1
(43) Pub. Date: Dec. 19, 2024

(54) NOVEL INNER CONTAINER OF SPERM EXTRACTOR

(71) Applicant: Zhejiang lianhong Technology Co., LTD, Huzhou (CN)

(72) Inventor: Xiu CHEN, Hangzhou (CN)

(73) Assignee: Zhejiang lianhong Technology Co., LTD, Huzhou (CN)

(21) Appl. No.: 18/513,929

(22) Filed: Nov. 20, 2023

(30) Foreign Application Priority Data
Jun. 19, 2023 (CN) 2023215713595

(51) Int. Cl. A61B 10/00; A61B 17/43; B65D 39/00; B65D 77/04

(52) U.S. Cl. CPC A61B 10/0058; A61B 17/43; B65D 39/0005; B65D 77/04

(57) ABSTRACT

Provided is a novel inner container of a sperm extractor, including: a soft rubber channel; and a butt joint assembly mounted at a tail end of the soft rubber channel, where the butt joint assembly includes a bracket, a circuit board, and a first housing, the bracket is embedded into the tail end of the soft rubber channel, the circuit board is mounted on the bracket, the first housing is located on an outer side of the circuit board and mounted at the tail end of the soft rubber channel, and three metal contacts are arranged on the circuit board and extend to an outer side of the first housing to be in butt joint with the sperm extractor.

### CROSS-REFERENCE TO RELATED APPLICATIONS

The application claims priority to Chinese patent application No. 2023215713595, filed on Jun. 19, 2023, the entire contents of which are incorporated herein by reference.

### TECHNICAL FIELD

The present utility model relates to the technical field of medical devices, and specifically to a novel inner container of a sperm extractor.

### BACKGROUND

With the continuous development of economy and society, people's life pressure is also increasing, leading to declined male sperm motility and increasingly prominent infertility. During doctor seeing in existing hospitals, it is often required to extract sperms within a short time and perform various tests on seminal fluids.

However, an existing medical sperm extractor, such as a sperm extractor provided in Chinese patent CN202121649564.X, cannot be replaced for its inner container, has poor sperm extraction, and may bring discomfort to patient's bodies.

Therefore, a new device needs to be provided to solve the above problems.

### SUMMARY

In view of the problems in the prior art, the present utility model provides a novel inner container of a sperm extractor.

To achieve the above objective, the present utility model adopts the following specific solution:

The present utility model provides a novel inner container of a sperm extractor, including:
a soft rubber channel; and
a butt joint assembly mounted at a tail end of the soft rubber channel,
where the butt joint assembly includes a first bracket, a circuit board, and a first housing,
the first bracket is embedded into the tail end of the soft rubber channel, the circuit board is mounted on the first bracket, the first housing is located on an outer side of the circuit board and mounted at the tail end of the soft rubber channel, and
three metal contacts are arranged on the circuit board and extend to an outer side of the first housing to be electrically connected to corresponding parts of a sperm extractor housing loaded with the inner container of the sperm extractor.

Further, the circuit board is further provided with an identification chip configured to identify a number of the inner container of the sperm extractor so as to activate a corresponding mode.

Further, two first magnets are further arranged in the first housing and configured to be in butt joint with the sperm extractor.

Further, an opening is formed in a front end of the soft rubber channel, and a barbed member is further arranged at the opening and configured to prevent internal liquid from flowing out.

Further, a second bracket is further arranged at an opening end of the soft rubber channel and embedded into the soft rubber channel.

Further, a fixing ring is further mounted on an outer side of the second bracket:
a clamping strip is arranged on a side wall of the second bracket: a clamping groove is correspondingly formed in an inner wall of the fixing ring, and the clamping strip rotates and then is clamped into the clamping groove to fix the fixing ring to the second bracket;
a second magnet is further mounted at a lower end of the fixing ring: and
a positioning strip is further arranged on an outer wall of the fixing ring.

Further, a third bracket is further mounted on the fixing ring:
a plurality of clamping columns are further arranged at a lower end of the third bracket: and a plurality of mounting holes are correspondingly formed in an upper end of the fixing ring, and the clamping columns are inserted into the mounting holes to fix the third bracket to the fixing ring.

Further, a waterproof rubber plug is further arranged at the tail end of the soft rubber channel through the circuit board and the first housing and configured to be taken down during cleaning so as to filter out remaining water inside.

With the technical solution of the present utility model, the following beneficial effects are achieved:
1. The three metal contacts are in butt joint with a sperm extractor body, making it convenient for replacement of different inner containers.
2. The inner container provided with the identification chip, making it convenient for the sperm extractor body to activate the corresponding mode based on information of the identification chip.
3. The inner container is provided with the magnets, making it convenient for automatic adsorption and butt joint with the sperm extractor body.
4. The barbed member is arranged at the opening to prevent the internal liquid from flowing out.

### BRIEF DESCRIPTION OF DRAWINGS

FIG. 1 is a three-dimensional view of the present utility model:

FIG. 2 is a sectional view of the present utility model:

FIG. 3 is an exploded bottom view of the present utility model; and

FIG. 4 is an exploded top view of the present utility model.

### IN THE DRAWINGS

101. soft rubber channel; 1011. barbed member; 1012. waterproof rubber plug;

102. first bracket; 103. circuit board; 1031. metal contact; 104. first housing; 1041. first magnet;

105. second bracket; 1051. clamping strip; 106. fixing ring; 1061. clamping groove; 1062. second magnet; 1063. mounting hole; 1064. positioning strip;

107. third bracket; and 1071. clamping column.

### DETAILED DESCRIPTION OF THE EMBODIMENTS

The present utility model is further described in detail below in conjunction with the accompanying drawings and embodiments. It may be understood that the specific embodiments described herein are used merely to explain the present utility model, rather than to limit the present utility model. It should be additionally noted that, for ease of description, only parts related to the present utility model are shown in the drawings.

In the description of the present utility model, unless otherwise expressly specified and limited, the terms “connected”, “connection”, and “fixed” should be understood in a broad sense. For example, it may be a fixed connection, a detachable connection, or integrated: it may be a mechanical connection or an electrical connection; and it may be directly connected, indirectly connected through an intermediate medium, internal communication between two components or an interactive relationship between two components. Those of ordinary skill in the art may understand specific meanings of the above terms in the present utility model according to specific circumstances.

In the present utility model, the state that the first feature is “over” or “under” the second feature may include a state that the first and second features are in direct contact with each other, or it may include a state that the first and second features are not in direct contact with each other and are in contact with each other via another feature between them, unless otherwise expressly specified and defined. Moreover, the state that the first feature is “over”, “above”, and “on” the second feature includes a state that the first feature is right above and obliquely above the second feature, or only indicates that the horizontal height of the first feature is greater than the horizontal height of the second feature. The state that the first feature is “under”, “below”, and “beneath” the second feature may include a state that the first feature is right below or obliquely below the second feature, or only indicates that the horizontal height of the first feature is less than the horizontal height of the second feature.

In the description of this embodiment, the orientations or positional relationships indicated by the terms “up”, “down”, “front”, “back”, “left”, “right”, etc. are based on the orientations or positional relationships shown in the accompanying drawings, only for the convenience of description and simplification of operation rather than indicating or implying that the apparatus or element referred to must have a particular orientation or be constructed and operated in a particular orientation, and therefore should not be construed as a limitation to the present utility model. In addition, the terms “first” and “second” are used merely to distinguish the descriptions and do not have special meanings.

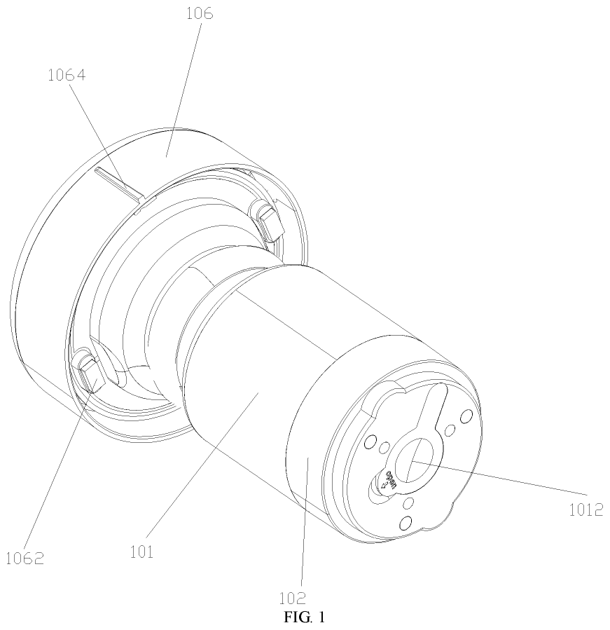

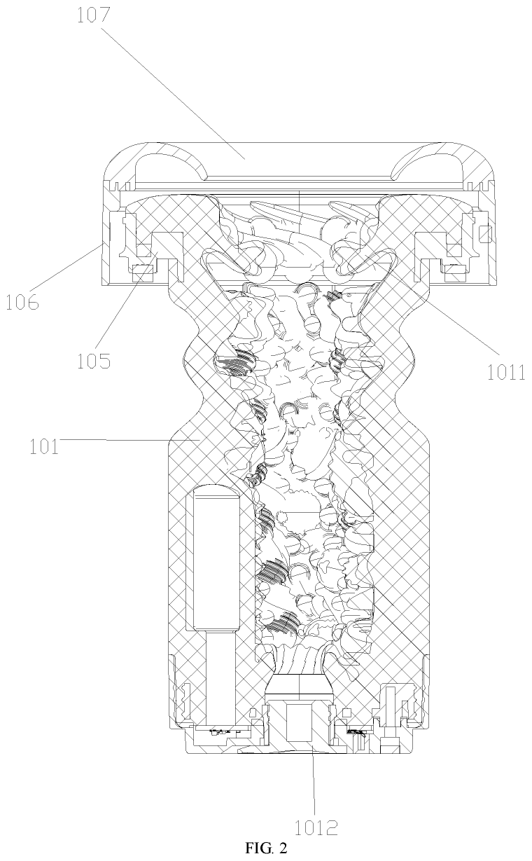

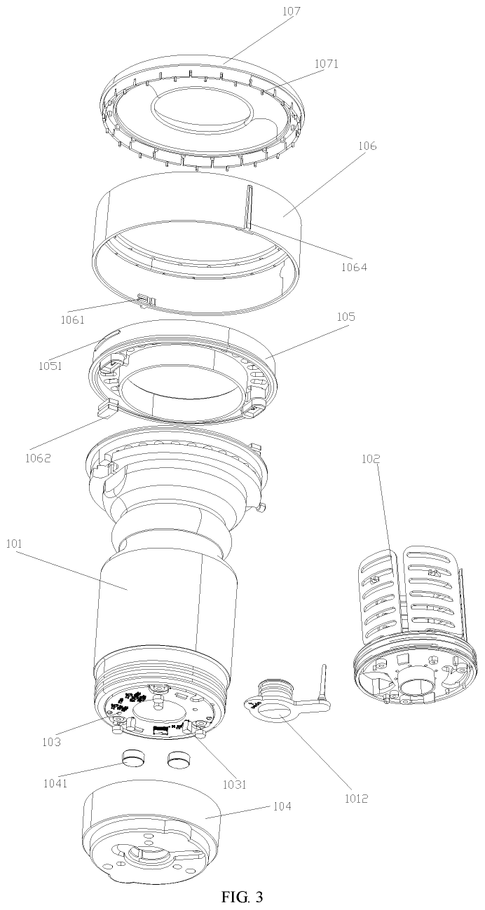

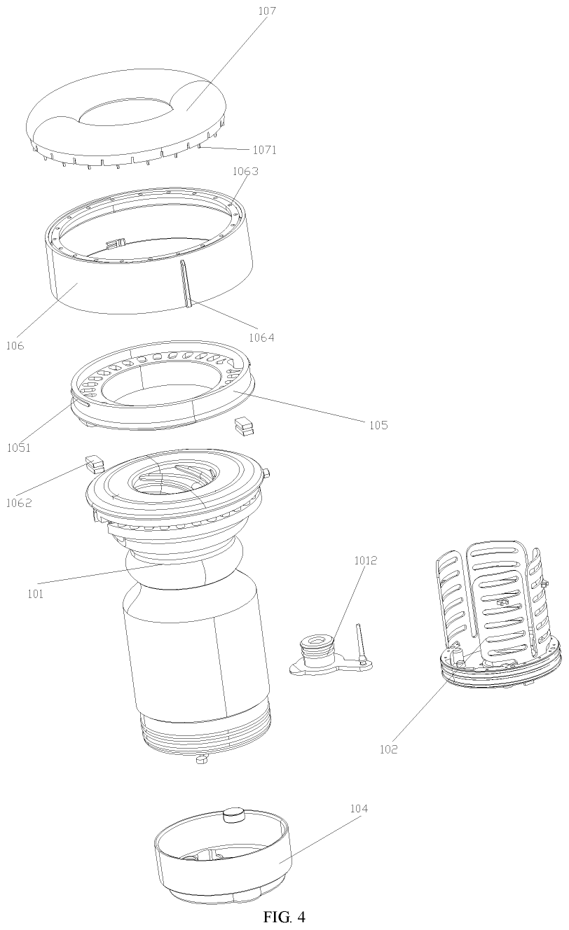

As shown in FIGS. 1 to 4, the present utility model provides a novel inner container of a sperm extractor, including: a soft rubber channel 101; and a butt joint assembly mounted at a tail end of the soft rubber channel 101, where the butt joint assembly includes a first bracket 102, a circuit board 103, and a first housing 104, the first bracket 102 is embedded into the tail end of the soft rubber channel 101, the circuit board 103 is mounted on the first bracket 102, the first housing 104 is located on an outer side of the circuit board 103 and mounted at the tail end of the soft rubber channel 101, and three metal contacts 1031 are arranged on the circuit board 103 and extend to an outer side of the first housing 104 to be electrically connected to corresponding parts of a sperm extractor housing loaded with the inner container of the sperm extractor.

The circuit board 103 is further provided with an identification chip configured to identify a number of the inner container of the sperm extractor so as to activate a corresponding mode.

Two first magnets 1041 are further arranged in the first housing 104 and configured to be in butt joint with the sperm extractor.

An opening is formed in a front end of the soft rubber channel 101, and a barbed member 1011 is further arranged at the opening and configured to prevent internal liquid from flowing out.

A second bracket 105 is further arranged at an opening end of the soft rubber channel 101 and embedded into the soft rubber channel 101.

A fixing ring 106 is further mounted on an outer side of the second bracket 105: a clamping strip 1051 is arranged on a side wall of the second bracket 105: a clamping groove 1061 is correspondingly formed in an inner wall of the fixing ring 106, and the clamping strip 1051 rotates and then is clamped into the clamping groove 1061 to fix the fixing ring 106 to the second bracket 105; a second magnet 1062 is further mounted at a lower end of the fixing ring 106; and
a positioning strip 1064 is further arranged on an outer wall of the fixing ring 106.

A third bracket 107 is further mounted on the fixing ring 106; a plurality of clamping columns 1071 are further arranged at a lower end of the third bracket 107; and a plurality of mounting holes 1063 are correspondingly formed in an upper end of the fixing ring 106, and the clamping columns 1071 are inserted into the mounting holes 1063 to fix the third bracket 107 to the fixing ring 106.

A waterproof rubber plug 1012 is further arranged at the tail end of the soft rubber channel 101 through the circuit board 103 and the first housing 104 and configured to be taken down during cleaning so as to filter out remaining water inside.

The principle of the present utility model is as follows:

The first bracket 102 is embedded into the tail end of the soft rubber channel 101, the circuit board 103 is mounted on the first bracket 102, the first housing 104 is located on the outer side of the circuit board 103 and mounted at the tail end of the soft rubber channel 101, and the three metal contacts 1031 are arranged on the circuit board 103 and extend to the outer side of the first housing 104 to be electrically connected to the corresponding parts of the sperm extractor housing loaded with the inner container of the sperm extractor, making it convenient for replacement of different inner containers.

The circuit board 103 is further provided with the identification chip, making it convenient for a sperm extractor body to activate the corresponding mode based on information of the identification chip.

Specifically, the three metal contacts 1031 of the inner container of the sperm extractor abut against and match with three elastic metal columns at the corresponding parts of the sperm extractor housing, and a control board of the external sperm extractor itself reads the number of the identification chip in the inner container of the sperm extractor and activates an action mode corresponding to the number.

The two first magnets 1041 are further arranged in the first housing 104, and the second magnet 1062 is further mounted at the lower end of the fixing ring 106, making it convenient for automatic adsorption and butt joint with the sperm extractor body.

The opening is formed in the front end of the soft rubber channel 101, and the barbed member 1011 is further arranged at the opening and configured to prevent the internal liquid from flowing out.

The above are only the preferred embodiments of the present utility model, rather than limiting the scope of patent of the present utility model. Any equivalent structure transformations made using the content of the specification and the accompanying drawings of the present utility model in a utility model conception of the present utility model, directly or indirectly applied in other related technical fields, are all included within the scope of protection of the present utility model.

What is claimed is:

1. A novel inner container of a sperm extractor, comprising:
a soft rubber channel; and
a butt joint assembly mounted at a tail end of the soft rubber channel,
wherein the butt joint assembly comprises a first bracket, a circuit board, and a first housing,
the first bracket is embedded into the tail end of the soft rubber channel, the circuit board is mounted on the first bracket, the first housing is located on an outer side of the circuit board and mounted at the tail end of the soft rubber channel, and
three metal contacts are arranged on the circuit board and extend to an outer side of the first housing to be electrically connected to corresponding parts of a sperm extractor housing loaded with the inner container of the sperm extractor.

2. The novel inner container of a sperm extractor according to claim 1, wherein the circuit board is further provided with an identification chip configured to identify a number of the inner container of the sperm extractor so as to activate a corresponding mode.

3. The novel inner container of a sperm extractor according to claim 1, wherein two first magnets are further arranged in the first housing and configured to be in butt joint with the sperm extractor.

4. The novel inner container of a sperm extractor according to claim 1, wherein an opening is formed in a front end of the soft rubber channel, and a barbed member is further arranged at the opening and configured to prevent internal liquid from flowing out.

5. The novel inner container of a sperm extractor according to claim 1, wherein a second bracket is further arranged at an opening end of the soft rubber channel and embedded into the soft rubber channel.

6. The novel inner container of a sperm extractor according to claim 5, wherein a fixing ring is further mounted on an outer side of the second bracket;
a clamping strip is arranged on a side wall of the second bracket; a clamping groove is correspondingly formed in an inner wall of the fixing ring, and the clamping strip rotates and then is clamped into the clamping groove to fix the fixing ring to the second bracket;
a second magnet is further mounted at a lower end of the fixing ring; and
a positioning strip is further arranged on an outer wall of the fixing ring.

7. The novel inner container of a sperm extractor according to claim 6, wherein a third bracket is further mounted on the fixing ring;
a plurality of clamping columns are further arranged at a lower end of the third bracket; and a plurality of mounting holes are correspondingly formed in an upper end of the fixing ring, and the clamping columns are inserted into the mounting holes to fix the third bracket to the fixing ring.

8. The novel inner container of a sperm extractor according to claim 1, wherein a waterproof rubber plug is further arranged at the tail end of the soft rubber channel through the circuit board and the first housing and configured to be taken down during cleaning so as to filter out remaining water inside.
